(19) United States
(12) Patent Application Publication
(10) Pub. No.: US 2024/0233110 A1
(43) Pub. Date: Jul. 11, 2024

(54) METHOD FOR DETECTING A POMELO FLESH MASS EDIBLE RATIO BASED ON X-RAY IMAGES

(71) Applicants: ZHEJIANG UNIVERSITY, Zhejiang (CN); ZHEJIANG KEPLER TECHNOLOGY CO., LTD., Zhejiang (CN)

(72) Inventors: Huirong XU, Zhejiang (CN); Yuchen ZHANG, Zhejiang (CN); Yibin YING, Zhejiang (CN); Lin LI, Zhejiang (CN)

(73) Assignees: ZHEJIANG UNIVERSITY, Zhejiang (CN); ZHEJIANG KEPLER TECHNOLOGY CO., LTD., Zhejiang (CN)

(21) Appl. No.: 18/559,563

(22) PCT Filed: Aug. 3, 2022
(86) PCT No.: PCT/CN2022/109950

§ 371 Date: Nov. 8, 2023

(30) Foreign Application Priority Data
Apr. 26, 2022 (CN) 202210449548.9

(51) Int. Cl. G06T 7/00; G06T 5/50; G06T 5/70; G06T 7/136

(52) U.S. Cl. CPC G06T 7/0004; G06T 5/50; G06T 5/70; G06T 7/136; G06T 2207/10116; G06T 2207/30128

(57) ABSTRACT

The disclosure provides a method for detecting a pomelo flesh mass edible ratio based on X-ray images: acquiring X-ray images A of a pomelo in a vertical direction and a transversal direction; performing noise reduction and grayscale stretching to obtain enhanced X-ray images B; processing the enhanced X-ray images B to obtain vertical and transversal 2D flesh masses; building a prediction model of an actual pomelo flesh mass; inputting the vertical and transversal 2D flesh masses into the prediction model to obtain a predicted flesh mass; weighting a fruit mass of the entire pomelo; and calculating a pomelo fruit edible ratio. The pomelo flesh mass is detected in a non-destructive manner in the disclosure through the combination of X-ray images and image processing technology, so rapid detection that saves time and labor costs is achieved. The accuracy of predicting the pomelo flesh mass is improved.

### TECHNICAL FIELD

The disclosure relates to a method for detecting a pomelo flesh mass edible ratio based on X-ray images and is mainly used in the post-harvest commercialization process of pomelos. The pomelos are graded according to their internal quality (flesh mass), so as to create a high-quality fruit brand effect and to improve the economic benefits of the pomelo industry.

### DESCRIPTION OF RELATED ART

China is a major pomelo producing country. China ranks first in the world in total production of pomelo, accounting for more than half of the global total. However, there is still much room for improvement in the economic benefits of China's pomelo industry. This is mainly because the technical means for post-harvest processing and commercialization of fruits in China are relatively less-advanced, and the market competitiveness is weak compared to many developed countries in the world. Many studies and practices have shown that the technology for detecting and grading the internal and external quality of fruits plays an important role in improving the economic benefits of the fruit industry.

Flesh mass is one of the important indicators indicating the internal quality of a pomelo. It is difficult to distinguish the mass of flesh only from the appearance, but the research on the methods for detecting the mass of pomelo flesh is very rare at home and abroad.

Problems such as thick skin and less-juicy flesh are found most of the time in pomelos with low flesh mass, which makes consumers unwilling to buy. The conventional manual measurement method is time-consuming and laborious. According to the national standard GB/T 27633-2011 of Guanxi honey pomelo, destructive methods are required for measurement, so it is difficult to satisfy the industrial needs of online detection.

A non-destructive, fast, and effective method for flesh mass detection is absent in the related art.

### SUMMARY

In order to solve the problems in the related art, the disclosure provides a method for detecting a pomelo flesh mass edible ratio based on X-ray images in which computerized image processing is used to detect flesh mass, and non-destructive, rapid, and effective flesh mass detection is achieved.

The technical solutions adopted by the disclosure are provided as follows.
1) Image acquisition: Through an X-ray image acquisition apparatus, original X-ray images A of a pomelo in two directions of a vertical direction and a transversal direction are acquired.
The transversal direction is parallel to the equatorial plane of the pomelo, and the vertical direction is perpendicular to the equatorial plane of the pomelo.
2) Image enhancement: Both noise reduction and grayscale stretching are performed on the acquired original X-ray images A in the two directions of the vertical direction and the transversal direction, and clear and high-contrast enhanced X-ray images B in the two directions of the vertical direction and the transversal direction are obtained.
3) Two-dimensional (2D) flesh mass acquisition: Both the vertical and transversal enhanced X-ray images B are processed, and a vertical 2D flesh mass and a transversal 2D flesh mass are respectively obtained.
4) Based on the vertical 2D flesh mass and the transversal 2D flesh mass, a prediction model using actual pomelo flesh mass is built for flesh mass prediction, a vertical 2D flesh mass and a transversal 2D flesh mass of a pomelo to be actually measured are inputted into the prediction model, and a predicted flesh mass is obtained.
5) A fruit mass of the entire pomelo is weighed next, and combined with the fruit mass of the entire pomelo, a pomelo fruit mass edible ratio is calculated: pomelo fruit mass edible ratio=predicted flesh mass/fruit mass of the entire pomelo.

In step 3), threshold segmentation is performed on both the vertical and transversal enhanced X-ray images B. Two predetermined thresholds are used to segment each image into three parts of a background area, a peel area (including a sponge layer), and a flesh area. Specific image processing is performed on the extracted flesh area to obtain a 2D flesh mass. 2D flesh masses obtained corresponding to the vertical and transversal enhanced X-ray images B are respectively treated as the vertical 2D flesh mass and the transversal 2D flesh mass.

In step 3), a grayscale value Gᵢ of each pixel in the flesh area is calculated as a sum of logarithms with e as a base as a 2D flesh mass of the pomelo, and the calculation formula is as follows:

$$2 D flesh mass = {Σ}_{i = 1}^{n} ln {G}_{i} ,$$

where Gᵢ represents the grayscale value of the i^{th} pixel in the flesh area, and n represents the total number of pixels in the flesh area.

The processing on the flesh area in the vertical enhanced X-ray image B and the processing on the flesh area in the transversal enhanced X-ray image B are the same.

After step 4), different grading ranges are set for the pomelo flesh mass in the method, and a flesh mass grade of the pomelo is determined according to the obtained predicted flesh mass.

The prediction model in step 4) is built according to the following formula:

y=A+Bx₁+Cx₂,

where y represents the predicted flesh mass, x1 represents the vertical 2D flesh mass, x2 represent the transversal 2D flesh mass, and A. B. and C respectively represents a first, second, and third fitting parameters of the prediction model.

The first, second, and third fitting parameters A. B. and C can all be obtained through pre-testing, calibration, and fitting.

An X-ray imaging system is used in the disclosure to acquire the original grayscale images of a pomelo in the vertical direction and the transversal direction. After noise reduction and grayscale stretching, clear and high-contrast X-ray images of the pomelo are obtained. The 2D flesh masses of the X-ray images of the pomelo are obtained next. Further, featuring the 2D flesh masses, a prediction model of the pomelo flesh mass is built, and the model can be used for the detection and classification of the pomelo flesh mass.

The beneficial effects provided by the disclosure include the following.

Non-destructive online detection is achieved in the disclosure through the combination of X-ray images and image processing technology, so rapid detection that saves time and labor costs is implemented. Further, the images in the two directions of the vertical direction and the transversal direction are introduced to further, so that the accuracy of predicting the pomelo flesh mass is further improved, and technical support for high-quality sorting of pomelos can be provided.

### BRIEF DESCRIPTION OF THE DRAWINGS

FIG. 1 is an overall flow chart according to the disclosure.

FIG. 2 is an original X-ray image of a pomelo in a vertical direction according to the disclosure.

FIG. 3 is an original X-ray image of the pomelo in a transversal direction according to the disclosure.

FIG. 4 is an enhanced image of FIG. 2 according to the disclosure.

FIG. 5 is an enhanced image of FIG. 3 according to the disclosure.

FIG. 6 is a scatter plot of a prediction model according to the disclosure.

### DESCRIPTION OF THE EMBODIMENTS

The disclosure is further described together with accompanying drawings and specific embodiments in the following paragraphs.

A pomelo is selected as an example in the disclosure.

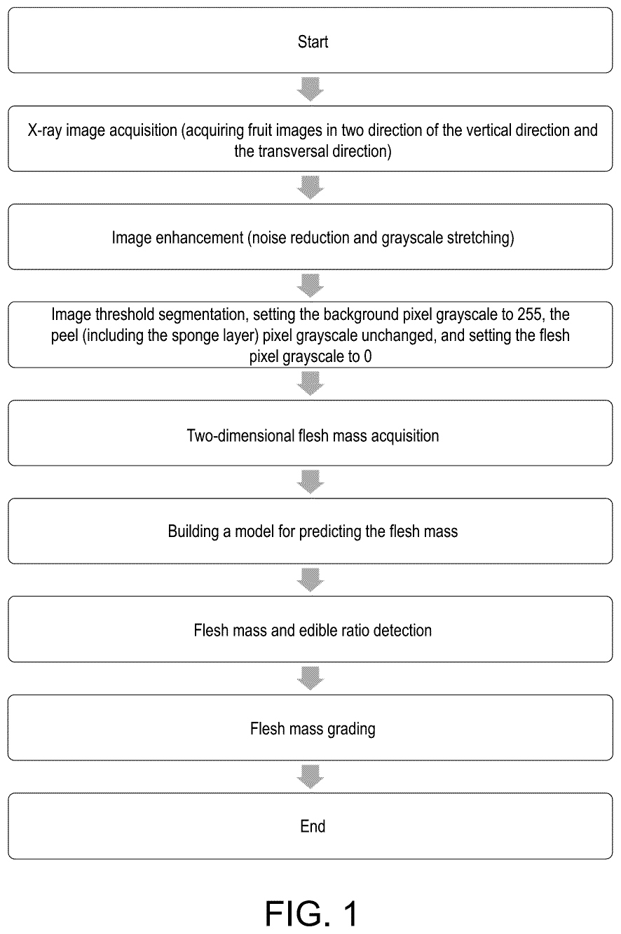

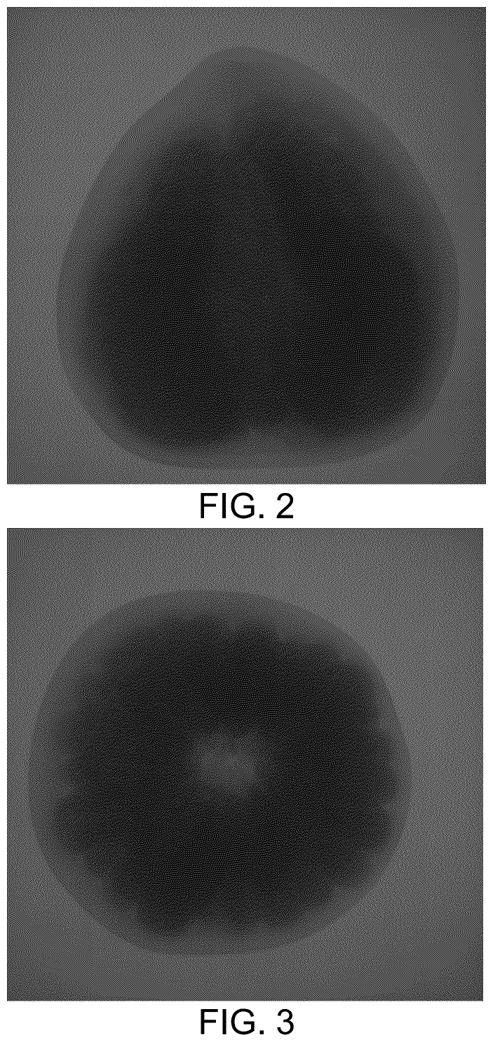

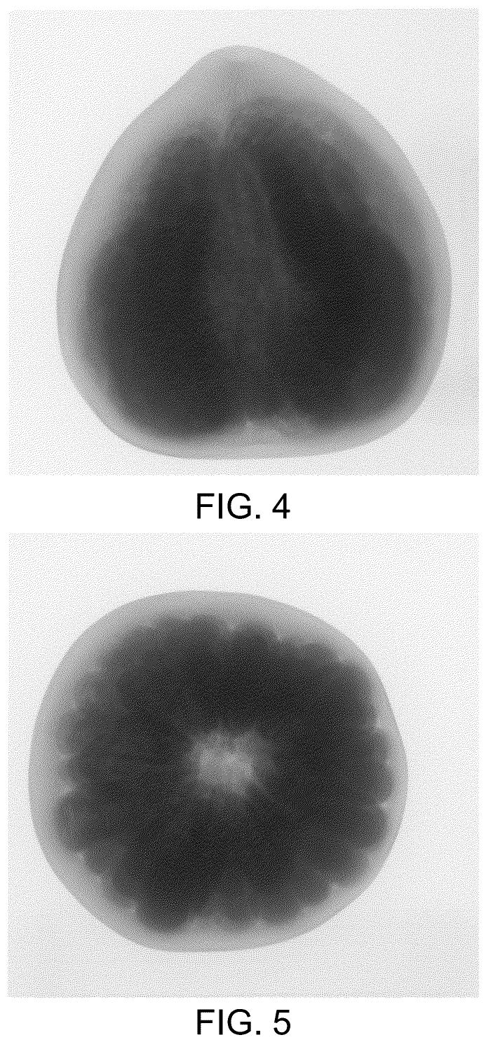

As shown in FIG. 1, the disclosure includes the following steps.
1) Image acquisition: As shown in FIG. 2 and FIG. 3, through an X-ray image acquisition apparatus, original X-ray images A of a pomelo in two directions of a vertical direction and a transversal direction are acquired.
2) Image enhancement: As shown in FIG. 4 and FIG. 5, both noise reduction and grayscale stretching are performed on the acquired original X-ray images A of the pomelo in the two directions. A Wiener filter with a window size of 9×9 is adopted for the noise reduction. A grayscale range of the transversal image A is linearly and smoothly stretched from 30-109 to 38-251, and a grayscale range of the vertical image A is linearly and smoothly stretched from 29-112 to 34-253 in the grayscale stretching method. Clear and high-contrast enhanced X-ray images B are obtained.
3) Two-dimensional (2D) flesh mass acquisition:
Adaptive threshold segmentation is performed on the enhanced X-ray images B. In the adaptive threshold segmentation method, a peak value I₁ whose grayscale value is closest to 255 is found first in a grayscale histogram corresponding to the enhanced X-ray images B. Then pixel points whose grayscale values are greater than I₁ are defined as the background, that is, the grayscale values are adjusted to 255. The proportion of each grayscale value in the foreground in a total matrix and a grayscale average value of the total foreground are calculated next. Starting from the grayscale value 0, a grayscale value I₂ is circularly searched to segment the foreground, and I₂ satisfies that the two parts after foreground segmentation have the largest inter-class variance. The pixel points whose grayscale values are less than I₂ are defined as a flesh area, the grayscale values remain unchanged, and the grayscale values of other pixels are adjusted to 0.
A sum of logarithms of a grayscale value (Gᵢ) of each pixel in the flesh area with e as a base is calculated (e.g., formula 1), which is the 2D flesh mass. The 2D flesh mass corresponding to FIG. 5 is 5.38×10⁶, and similarly, the 2D flesh mass of the pomelo obtained from FIG. 4 is 4.51×10⁶;

$$2 D pulp content = {Σ}_{i = 1}^{n} ln {G}_{i} \qquad ( 1 )$$

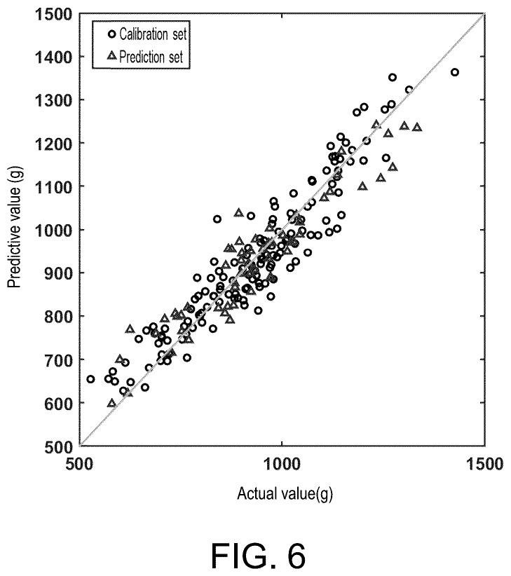

4) As shown in FIG. 6, a total of 98 Hongrou honey pomelos and 104 Guanxi honey pomelos are selected for the measurement of the vertical 2D flesh mass and transversal 2D flesh mass. A calibration set and a prediction set respectively account for 70% and 30% of the total number of pomelos. A multiple linear regression model (e.g., formula 2) for the actual flesh masses of the pomelos is established. A coefficient of determination Rₚ² of the prediction set is 0.888, a root-mean-square error of the prediction set (RMSEP) is 57.43 g, and a residual predictive deviation (RPD) of the model is 2.87;

y=−107.4210+x₁×8.9658×10⁻⁵+x₂×1.1204×10⁻⁴ (2),

where, y represents a predicted flesh mass of a pomelo, x₁ represents a grayscale logarithmic sum of the flesh area of the vertical X-ray image, and x₂ represents a grayscale logarithmic sum of the flesh area of the transversal X-ray image.
5) Flesh mass grading of the pomelos: In specific implementation, the pomelo flesh masses greater than 1100 g are set as grade 1, those within the range of 700 g to 1100 g are as grade 2, and those less than 700 g are set as grade 3. The flesh masses of the pomelos predicted and obtained in step 4) are graded.
6) Combined with a fruit weight of an entire pomelo obtained by weighing, a fruit mass edible ratio of the pomelo, that is, predicted flesh mass/entire fruit mass, can also be calculated.

It thus can be seen that in the disclosure, the detection and grading of the flesh mass of pomelos can be achieved, and the problems of long detection hours, labor consumption, and destruction found in the conventional detection methods may be solved. Rapid and non-destructive detection saving much time and labor costs is provided, the benefits generated for the pomelo industry may thus be improved, and the detection of edible ratio is also implemented.

The above description is only preferred embodiments of the disclosure and is not intended to limit the disclosure. All minor modifications, equivalent replacements, and improvements made to the above embodiments according to the technical essence of the disclosure shall be included in the protection scope of the technical solutions of the disclosure.

What is claimed is:

1. A method for detecting a pomelo flesh mass edible ratio based on X-ray images, wherein the method comprises following steps:
1) image acquisition: acquiring, through an X-ray image acquisition apparatus, original X-ray images A of a pomelo in two directions of a vertical direction and a transversal direction;
2) image enhancement: performing both noise reduction and grayscale stretching on the acquired original X-ray images A in the two directions of the vertical direction and the transversal direction, and obtaining enhanced X-ray images B in the two directions of the vertical direction and the transversal direction;
3) two-dimensional (2D) flesh mass acquisition: processing both the vertical and transversal enhanced X-ray images B and respectively obtaining a vertical 2D flesh mass and a transversal 2D flesh mass;
4) based on the vertical 2D flesh mass and the transversal 2D flesh mass, building a prediction model of an actual pomelo flesh mass, inputting a vertical 2D flesh mass and a transversal 2D flesh mass of a pomelo to be actually measured into the prediction model, and obtaining a predicted flesh mass; and
5) weighing a fruit mass of the entire pomelo next and calculating, combined with the fruit mass of the entire pomelo, a pomelo fruit mass edible ratio: pomelo fruit mass edible ratio=predicted flesh mass/fruit mass of the entire pomelo.

2. The method for detecting the pomelo flesh mass edible ratio based on the X-ray images according to claim 1, wherein in step 3), threshold segmentation is performed on both the vertical and transversal enhanced X-ray images B, predetermined thresholds are used to segment each image into three parts of a background area, a peel area, and a flesh area, specific image processing is performed on the extracted flesh area to obtain a 2D flesh mass, and 2D flesh masses obtained corresponding to the vertical and transversal enhanced X-ray images B are respectively treated as the vertical 2D flesh mass and the transversal 2D flesh mass.

3. The method for detecting the pomelo flesh mass edible ratio based on the X-ray images according to claim 1, wherein in step 3), a grayscale value Gᵢ of each pixel in the flesh area is calculated as a sum of logarithms with e as a base as a 2D flesh mass of the pomelo, and the calculation formula is as follows;
$$2 D flesh mass = {\sum }_{i = 1}^{n} ln {G}_{i}$$
wherein Gᵢ represents the grayscale value of the i^{th} pixel in the flesh area, and n represents the total number of pixels in the flesh area.

4. The method for detecting the pomelo flesh mass edible ratio based on the X-ray images according to claim 1, wherein after step 4), different grading ranges are set for the pomelo flesh mass in the method, and a flesh mass grade of the pomelo is determined according to the obtained predicted flesh mass.
